(19) United States
(12) Patent Application Publication
(10) Pub. No.: US 2008/0096793 A1
(43) Pub. Date: Apr. 24, 2008

(54) Class III Slrp for the Treatment of Cancer

(75) Inventors: Paul N. Bishop, Manchester (GB); Rachel L. Cowen, Manchester (GB)

Correspondence Address: MICHAEL BEST & FRIEDRICH LLP, 100 E WISCONSIN AVENUE, Suite 3300, MILWAUKEE, WI, 53202, US

(73) Assignee: THE UNIVERSITY OF MANCHESTER, Manchester (GB)

(21) Appl. No.: 11/577,658

(22) PCT Filed: Oct. 13, 2005
(86) PCT No.: PCT/GB05/03940

§ 371 Date: Apr. 20, 2007

(30) Foreign Application Priority Data
Oct. 20, 2004 (GB) 0423273.2

(51) Int. Cl. A61K 48/00; A61K 38/17

(52) U.S. Cl. 514/2; 514/44

(57) ABSTRACT

There is provided the use of an agent that promotes class III SLRP activity in the manufacture of a medicament for the prevention and/or treatment of cancer. Such medicaments may be used in the treatment of tumours, including avascular tumours. Suitable agents to be used may preferably include the class III SLRPs opticin; epiphycan; or mimecan. The invention also provides a method for the prevention and/or treatment of cancer.

The present invention relates to medicaments for the treatment and/or prevention of cancer. More particularly it relates to medicaments for the early treatment of cancer, and for the prevention of tumour cell proliferation.

Cancer constitutes one of the greatest causes of mortality in the Western hemisphere. It is estimated that one in three adults in Great Britain will suffer from cancer at some point in their lives.

Approximately two hundred different types of cancer are recognised. The prevalence of these different types of cancers varies widely, and cancers of the lung, breast, bowel and prostate account for over half of all new cases diagnosed.

The progression of cancer development can generally be thought of as progressing sequentially through two distinct phases, the avascular phase and vascular phase. The avascular phase represents the earliest stage of tumour development. During the avascular phase the tumour comprises a nucleus of transformed cells, but is not provided with a supply of blood vessels. The size of tumour that may develop during the avascular phase is limited by the requirement for oxygen and nutrient uptake to take place by diffusion alone.

Tumour development may then progress into the vascular phase. In this phase the tumour develops its own blood supply, usually by angiogenic sprouting from existing blood vessels. The development of a dedicated blood supply allows the tumour to increase markedly in size, since the nutrient and oxygen requirements of the transformed cells may be met through the new blood vessels supplying the cancer.

In addition to allowing an increase in tumour size, the transition from the avascular to vascular phase correlates strongly with the onset of tumour metastasis and increased tumour invasiveness. It will be readily appreciated that this change to a metastatic phenotype marks a dangerous development in cancer progression.

Many present cancer therapies target the development of tumour vasculature as a means by which tumour development and progression may be retarded or prevented. Accordingly there is significant interest in the clinical application of anti-neovascular and anti-angiogenic approaches to cancer therapy. However, even in the light of recent developments in cancer therapies, there still remains a requirement for the development of further therapies able to replace or augment those already in existence.

Furthermore, it will be recognised that, since the progression to the vascular phase is associated with metastasis and tumour dissemination, it is advantageous to treat the developing tumour before the vascular phase is reached. Although anti-neovascular approaches may help prevent vascularisation and metastasis it may be preferred to treat the tumour before the risk of metastasis arises. Such treatments able to directly influence and inhibit proliferation and/or viability of cancer cells may be used at earlier time-points in tumour development and progression than anti-neovascular therapies, and may be effective during the relatively less damaging avascular phase.

In the light of the preceding paragraphs it will be recognised that there exists a well-established need for the development of new medicaments and methods of treatment for the prevention and/or treatment of cancers. Furthermore, there is a recognised need for medicaments and methods of treatment suitable for the prevention and/or treatment of cancers in the avascular phase in order to allow treatment to start from the earliest possible time-point, thereby reducing the risk of metastasis.

According to a first aspect of the present invention there is provided the use of an agent that promotes class III small leucine-rich repeat protein/proteoglycan (class III SLRP) activity in the manufacture of a medicament for the prevention and/or treatment of cancer.

According to a second aspect of the invention there is provided a method of preventing and/or treating cancer, the method comprising administering a therapeutically effective amount of an agent that promotes class III small leucine-rich repeat protein/proteoglycan (class III SLRP) activity to an individual in need of such prevention and/or treatment.

The inventor has discovered that members of the class III small leucine-rich repeat protein/proteoglycan (SLRP) family, such as opticin (a class III SLRP that was first identified associated with collagen fibrils in the vitreous humour, and which is also known as oculoglycan), epiphycan, mimecan (which is also known as osteoglycin) are able to inhibit the proliferation and/or viability of cancer cells, and/or to promote apoptosis of cancer cells. As will be appreciated, these properties make such agents suitable for use in the prevention and/or treatment of cancer. This finding has been established in cancer cells derived from a number of different contexts, including fibrosarcomas, breast cancers and lung cancers.

The present invention is particularly advantageous in that it allows the prevention and/or treatment of cancer when the cancer is in the avascular phase. Accordingly the cancer may be treated earlier in its development, and particularly before progression into the vascular phase (a progression associated with development of a metastatic phenotype). It is preferred that the cancer to be treated in accordance with the first or second aspects of the invention is a tumour, and more preferred that the tumour is an avascular tumour.

The cancer to be prevented and/or treated may suitably be selected from the group comprising a fibrosarcoma, a glioma, pancreatic cancer, bladder cancer, colon cancer, breast cancer and lung cancer.

The term “agent that promotes class III SLRP activity” as used in the present application encompasses class III SLRPs per se; biologically active fragments of class III SLRPs; derivatives of class III SLRPs; and also agents that mimic class III SLRP activity.

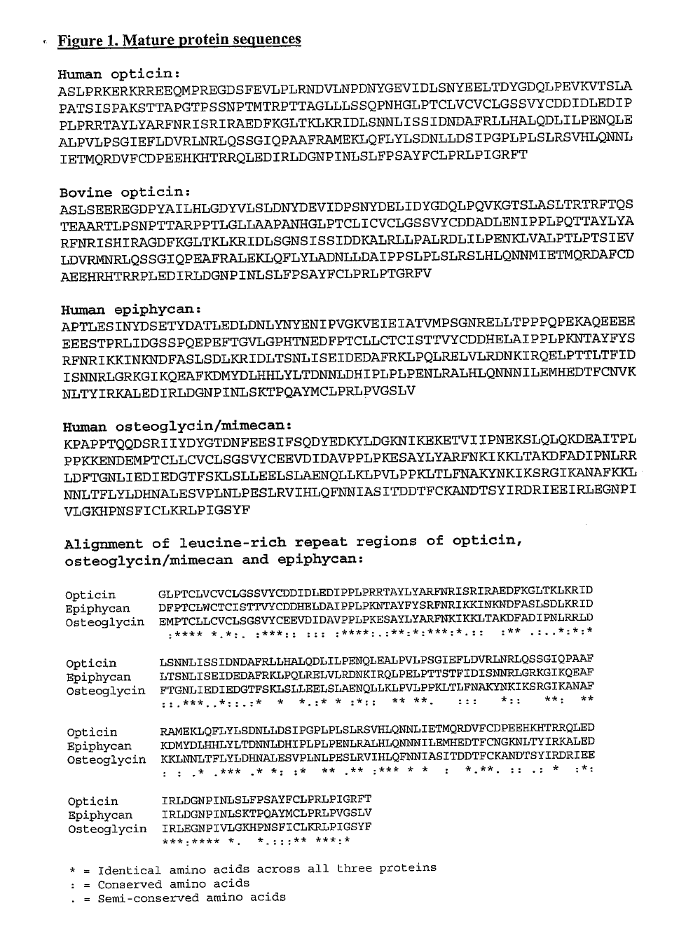

The amino acid sequences of opticin (both human and bovine forms), epiphycan and mimecan are shown in FIG. 1. This Figure also shows alignment of the amino acid sequences, illustrating the high degree of similarity between different members of the class III SLRP family.

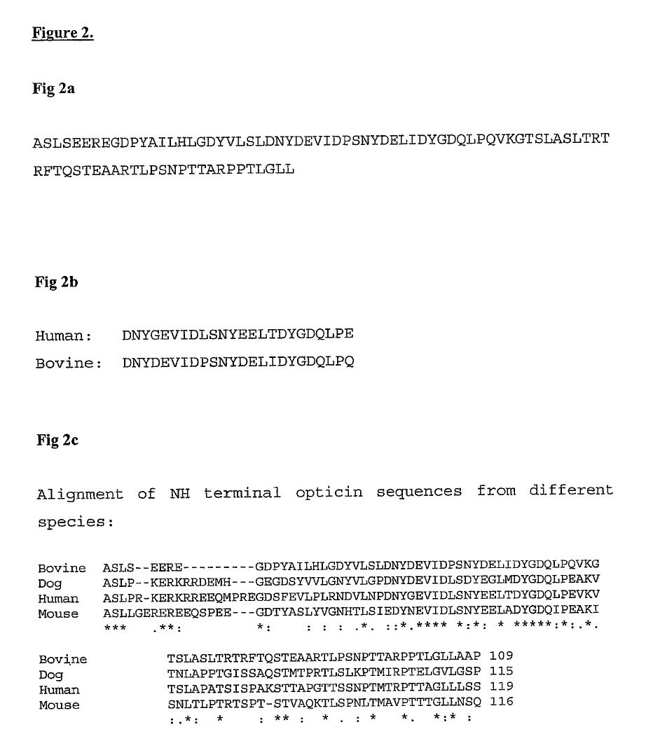

FIG. 2 illustrates (in panel 2a) a biologically active fragment released on cleavage of the class III SLRP opticin with matrix metalloproteinases 2 or 9 (MMP-2 or MMP-9). Panel 2b shows peptide sequences from within the NH terminal region of human and bovine opticin that are preferred for use as agents in accordance with the invention. These sequences are highly conserved between species, this great degree of conservation being indicative of their biological function. Panel 2c of FIG. 2 illustrates alignment of amino acid residues in the NH terminal region of opticin derived from different species (cow, dog, human and mouse).

Class III SLRPs, modified forms of class III SLRPs, and biologically active fragments thereof, are able to inhibit proliferation and/or viability of cancer cells. Accordingly, it will be appreciated that class III SLRPs, their fragments and derivatives have utility in the prevention and/or treatment of cancer. Although we do not wish to be bound by any hypothesis the inventor believes that class III SLRPs are able to induce cancer cells, which are normally predisposed to elevated, and indeed uncontrolled, proliferation to stop proliferating and/or to undergo apoptosis.

Agents used according to the first and second aspects of the invention may be any compound or composition that mimics the effect of class III SLRPs in vivo. However, it is preferred that the agent is:

a) opticin (also known as oculoglycan); or

b) epiphycan; or

c) mimecan (also known as osteoglycin); or

d) a chimeric molecule comprising elements of any of a) to c);

e) a modified form of any of a) to d); or

f) a biologically active fragment or derivative of any of a) to e).

It will be appreciated that agents suitable for use according to the invention encompass molecules able to mimic the activity of class III SLRPs. Such molecules may be capable of replicating the binding activity of class III SLRPs (e.g. capable of binding to cells that are targets of class III SLRP binding). Preferred molecules may, for example, replicate the conformation of portions of class III SLRPs that are important in achieving their biological functions. Suitable agents capable of mimicking class III SLRP activity may include small soluble molecules.

It will further be appreciated that the binding properties of synthetic or natural agents suitable for use according to the invention may be modified, in accordance with the use to which the agent is to be put, to produce improved agents. For instance, agents to be used in accordance with the first or second aspects of the invention may be modified in order to increase their ability to bind to cancer cells. Many methods suitable for the targeting of agents to cancer cells are known to those of skill in the art, and these may be applied to the development, design and production of agents comprising, or based on, class III SLRPs. Examples of such targeting methods include the combination of agents according to the invention with antibodies able to specifically bind to epitopes associated with cancer cells. Suitable examples of such epitopes are well known to those skilled in the art.

Similarly, agents for use in accordance with the first or second aspects of the invention may be modified to increase their ability to bind receptors associated with cancer cells. Examples of suitable receptors that may form the basis of such targeting modifications include integrins, cell surface proteoglycans and growth factor receptors. Suitable receptors in each of these classes will be readily apparent to the skilled person.

The agent is preferably a human class III SLRP, or a fragment or derivative thereof. Most preferably the agent is human opticin or a fragment or derivative thereof.

In the event that the agent is a non-human class III SLRP, or a fragment or derivative thereof, it is preferred that the agent be one that is well tolerated in a human patient to which the agent is administered. A suitable non-human derived agent may be selected such that it contains few epitopes likely to contribute to the agent's rejection by a human patient, or may be “humanised” for example by modification to include portions of the corresponding human class III SLRP sequence. A preferred example of such an agent derived from a non-human class III SLRP is one derived from a bovine class III SLRP, such as bovine opticin.

Class III SLRPs may be isolated from naturally occurring sources. By way of example, the class III SLRP opticin is abundant in the vitreous humour of the eye, and accordingly may be enriched and isolated from this tissue. Alternatively class III SLRPs and their derivatives may be produced using recombinant DNA technologies. Preferably the agent may be a recombinant class III SLRP or a fragment or derivative thereof. Recombinant class III SLRPs and their derivatives maybe produced from many alternate sources, for instance bovine recombinant class III SLRPs such as opticin. More preferably such agents comprise a recombinant human class III SLRP or fragment or derivative thereof. Most preferably the agent is recombinant human opticin, or a fragment or derivative thereof.

The term “biologically active fragment of a class III SLRP” as used according to the invention encompasses fragments, which are able to replicate SLRPs' activities (in terms of their ability to inhibit cancer cell proliferation and/or viability, and preferably to induce apoptosis of cancer cells) as assessed by either in vivo or in vitro assays. Similarly, references to “modified forms” of agents of the invention should be taken to relate to those modified forms that retain the required biological activity. Suitable assays by which the biological activity of fragments, derivatives or modified forms of a class III SLRPs (and thereby the suitability of such fragments, derivatives or modified forms for use in accordance with the invention) may be assessed include those set out in the Examples section below. For example, suitable biological activity of fragments, derivatives or modified forms for use in accordance with the invention may be ascribed to those fragments, derivatives or modified forms that are able to inhibit tumour cell proliferation in vivo or in vitro.

It is known that the class III SLRP opticin comprises a homodimer formed by non-covalently linked leucine-rich repeats (LRRs) linked to an amino-terminal (NH) domain. The LRR domain and NH domain may be enzymatically cleaved from one another, and such enzymatically cleaved class III SLRP fragments represent a preferred agent for use in accordance with the first and second aspects of the invention. Preferably enzymatic cleavage of class III SLRPs such as opticin may be undertaken using the matrix metalloproteinases MMP-2, MMP-9 or MMP-12. MMP-12 is a matrix metalloproteinase known to be implicated in the growth and progression of tumours. By way of example, the amino acid sequence of a NH terminal fragment derived by MMP-2 or MMP-9 cleavage of bovine opticin is illustrated in FIG. 2. The skilled person will appreciate that similar cleavage products may be produced on enzyme treatment of human class III SLRPs.

The NH domain fragment of enzymatically cleaved class III SLRPs are soluble and represent preferred agents suitable for use according to the invention. The NH domain may preferably be the NH domain of opticin.

Alternatively N-terminal fragments of class III SLRPs that have been derived by means other than enzymatic digestion may also be used as agents in accordance with the invention. By N-terminal fragment is meant any fragment comprising at least seven contiguous amino acids, preferably at least twelve contiguous amino acid residues, and more preferably at least twenty-four contiguous amino acid residues, from within the sixty-five amino acids located the N-terminal of a class III SLRP.

A preferred N-terminal fragment suitable for use in accordance with the invention may comprise one of the following amino acid sequences taken from the human and bovine forms of opticin:

| | |
|---|---|
| From human opticin: | DNYGEVIDLSNYEELTDYGDQLPE |
| From bovine opticin: | DNYDEVIDPSNYDELIDYGDQLPQ |

The above sequences are highly conserved between species, indicative of the importance of these amino acid residues in mediating class III SLRPs' biological activities.

Fragments of class III SLRPs other than those derived by enzyme cleavage may, for example, be produced by any suitable peptide synthesis methodology known in the art. The constituent amino acids of such synthesised fragments may be selected to include preferred portions of the class III SLRP sequence. The skilled person will appreciate that the range of possible non-enzymatically derived fragments of class III SLRPs is greater than that which may be derived by enzyme digest, since the potential sequences of such synthesised fragments are not constrained by the location of enzyme cleavage sites within the class III SLRP.

It will be appreciated that LRR regions of class III SLRPs may also represent suitable agents for use in accordance with the invention. The LRR region may suitably be utilised in the form of fragments including the LRR region of class III SLRPs. Such fragments may additionally comprise further amino acid residues from the C-terminal portion of a suitable class III SLRP. Fragments may be derived enzymatically or by other means, in the same way as considered above.

Preferably the LRR domain is the LRR of opticin, however the LRR domains of epiphycan and mimecan also represent suitable agents for use in accordance with the first and second aspects of the invention, since these LRR domains are also known to be glycosylated and to have relatively high solubility. LRR regions of the different class III SLRPs share a high degree of similarity with one another, as illustrated by the sequence alignment data shown in FIG. 1, and thus the skilled person would recognise that the biological activities associated with the LRR region of a particular class III SLRP may be expected to be common to other members of the family.

It will be appreciated that preferred chimeric molecules suitable for use as agents of the invention may be those that comprise one or more of the fragments, domains or regions of interest considered in the preceding paragraphs. The possession of biological activity indicating the suitability of such chimeric molecules for use in accordance with the invention may be investigated using the techniques detailed elsewhere in the specification.

The ability of agents according to the invention to inhibit cancer cell proliferation may be readily assessed using methods that will be familiar to the skilled person. Suitable methods include comparing rates of proliferation in populations of cancer cells grown in the presence or absence of a putative active agent. Comparison of the rates of proliferation exhibited by such test populations will allow ready assessment of the ability of the putative active agent to inhibit cancer cell proliferation.

Similarly, the ability of agents according to the invention to decrease cancer cell viability may be investigated using techniques well known to those of skill in the art. For example, cancer cells may be contacted with a putative agent according to the invention and their viability assessed by means of the trypan blue exclusion assay. Cancer cell viability values derived in this way may be compared with reference values to assess the effectiveness of the putative agent in decreasing cancer cell viability.

The ability of agents according to the invention to promote cancer cell apoptosis may also be readily assessed using methods well known to those of skill in the art. In vitro or in vivo populations of cancer cells may be exposed to a putative active agent, and the extent of apoptosis occurring investigated using suitable techniques. Examples of techniques by which apoptosis may be investigated histological stains such as acridine orange, or labelling techniques such as TUNEL (Transferase-mediated dUTP Nick-End Labelling).

Derivatives of class III SLRPs, or fragments thereof, may include derivatives of class III SLRPs that increase or decrease class III SLRPs' half-lives in vivo. Examples of derivatives that increase the half-life of class III SLRPs, or class III SLRP fragments, include modified class III SLRPs in which enzyme cleavage motifs have been removed by amino acid deletion and/or substitution, peptoid derivatives of class III SLRPs, D-amino acid derivatives of class III SLRPs and peptide-peptoid hybrids.

Agents such as native class III SLRPs, modified class III SLRPs or class III SLRP fragments, may be subject to degradation by a number of means (such as protease activity in biological systems). Such degradation may limit the bioavailability of class III SLRPs (or their fragments), and hence the ability of class III SLRPs to prevent and/or treat cancer. There are many examples of well-established techniques by which peptide derivatives that have enhanced stability in biological contexts can be designed and produced. Such peptide derivatives may have improved bioavailability as a result of increased resistance to protease-mediated degradation.

Preferably a peptide derivative or analogue suitable for use according to the invention is more protease-resistant than the peptide (or glycoprotein) from which it is derived. Suitable methods by which protease-resistance may be conferred include protection, substitution or modification of serine or threonine residues present in class III SLRPs. Protease-resistance of a peptide derivative and the peptide (or glycoprotein) from which it is derived may be evaluated by means of well-known protein degradation assays. The relative values of protease resistance for the peptide derivative and peptide (or glycoprotein or proteoglycan) may then be compared.

Peptoid derivatives of the agents of the invention may be readily designed from knowledge of the structure of class III SLRPs. Commercially available software may be used to develop peptoid derivatives according to well-established protocols.

Retropeptoids, (in which all amino acids are replaced by peptoid residues in reversed order) are also able to mimic a high-affinity binding proteins. A retropeptoid is expected to bind in the opposite direction in the ligand-binding groove, as compared to a peptide or peptoid-peptide hybrid containing one peptoid residue. As a result, the side chains of the peptoid residues are able point in the same direction as the side chains in the original peptide.

A further embodiment of a modified form of class III SLRPs suitable for use according to the invention comprises D-amino acids. In this case the order of the amino acid residues is reversed as compared to that found in the native class III SLRP.

It will be appreciated that derivatives of class III SLRPs suitable for use in accordance with the invention also include modified forms of class III SLRPs, or fragments thereof, in which the amino acid sequence has been altered compared to that of the corresponding native class III SLRP. These modified or variant forms of class III SLRPs may be produced by the addition, subtraction or substitution of one or more of the amino acid residues occurring in the native molecule. Suitable methods by which such addition, subtraction or substitution variants may be produced are well known to those skilled in the art, and are the subject of a great number of publications which provide details of experimental protocols which may be used. The nature of the amino acid substitutions to be made may be determined with reference to the effect that it is desired to achieve. For example, as discussed above, modified forms of class III SLRPs may be designed to remove enzyme cleavage sites, thereby reducing enzymatic degradation and increasing half-life in vivo. There exists a wealth of publicly available information regarding amino acid motifs digested by different proteolytic enzymes and the skilled person would readily be able to recognise the occurrence of such motifs within native class III SLRP molecules. It is then a straightforward matter to produce modified versions of class III SLRPs in which amino acids are added, removed or replaced in order to disrupt the cleavage site.

Modified forms of class III SLRPs may also be produced such that they include amino acid sequences that may interact advantageously with the local environment to achieve a desired effect. For instance amino acid sequences may be introduced in order to promote binding of the modified class III SLRP to components of the extracellular matrix (ECM), thereby providing a reserve of the modified class III SLRP available in the vicinity of cells the activity of which it is wished to influence. For instance the skilled person may modify class III SLRPs so that they include amino acid sequences that promote adhesion of the modified molecules to ECM components associated with cancer cells and tumour development.

Preferably modifications of class III SLRP sequence by addition or substitution of amino acids may be conservative modifications, such that the tertiary structure of the variant is not significantly altered from that of the native class III SLRP from which the variant is derived. There exists in the scientific literature a wealth of information to assist the skilled person in the production of modified peptides with conservative additions or substitutions, and the appropriate selection of amino acid residues to achieve such conservative modifications does not require the application of inventive activity on the part of the skilled practitioner.

Preferably a variant form of a class III SLRP may share at least 50% amino acid sequence identity with the corresponding portion of the native class III SLRP from which it is derived. More preferably the degree of identity may be at least 60%, or 70%, and most preferably the variant may share at least 80%, 90% or 95% homology with the corresponding portion of the sequence of the native peptide from which it is derived. The similarity of the amino acid sequence of variant forms of class III SLRPs to native molecules may readily be determined using freely available comparison software.

It will be appreciated that the agents according to the present invention may be used in a monotherapy (i.e. use of agents according to the invention alone to prevent and/or treat cancer).

Alternatively agents according to the invention may be used as an adjunct, or in combination with, known therapies for the prevention and/or treatment of cancer. The use of agents according to the invention in combination with other therapies may be preferred, since it is generally believed to be advantageous to activate multiple pathways capable of bringing about the inhibition or destruction of cancer cells. The advantages of such combined therapies may include more rapid resolution of cancer prevention and/or treatment, reduced danger of the development of a metastatic phenotype, and reduction of the risk of the cancer cells developing resistance to treatment.

Suitable known therapies for the prevention and/or treatment of cancer that may be used in combination in accordance with agents of the invention include chemotherapy, radiotherapy, therapy employing agents such as taxol, and anti-angiogenic therapies (which may advantageously prevent or delay progression of tumours to be treated to the vascular phase).

In a third aspect of the invention there is provided the combination of an agent capable of promoting class III SLRP activity and a further cancer therapy agent. Preferably the further cancer therapy agent may be a chemotherapeutic agent, an anti-angiogenic agent, or taxol. The combination of the agent of the invention and the further cancer therapy agent may be provided in the form of an admixture, or in separate dosage forms.

Agents according to the invention may be used in combination with substances capable of inhibiting integrin function. Such substances may include neutralising antibodies capable of binding to integrins. Preferably the integrins the function of which is to be inhibited may be selected from the group comprising α4, α5, αV, β1 and β3.

Agents according to the invention may be combined in compositions having a number of different forms depending, in particular on the manner in which the composition is to be used. Thus, for example, the composition may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given.

Compositions comprising agents according to the invention may be used in a number of ways. For instance, systemic administration may be required in which case the compound may be contained within a composition which may, for example, be ingested orally in the form of a tablet, capsule or liquid. Alternatively the composition may be administered by injection into the blood stream. Injections may be intravenous (bolus or infusion) or subcutaneous (bolus or infusion). The compounds may be administered by inhalation (e.g. intranasally), a route of administration that may be preferred in the case of the prevention and/or treatment of lung cancer.

Compositions comprising agents according to the invention may be used to prevent and/or treat ocular cancers. Such compositions may be formulated for injection, either into the eye itself (e.g. intravitreal injection) or around the eye (e.g. peri-orbital injection). Alternatively, compounds may be formulated for topical application or irrigation of the eye, for instance in the form of eyedrops. Suitable compositions of formulations for injection or topical use will be well known to those skilled in the art.

Iontophoresis represents another route by which agents according to the invention may be delivered to a desired tissue. Iontophoresis may provide a preferred method by which agents may be introduced non-invasively to a site at which it is wished to prevent and/or treat cancer.

Agents may also be incorporated within a slow or delayed release device. Such devices may, for example, be inserted on or under the skin, or other tissues and the compound may be released over weeks or even months. Such devices may be particularly advantageous when long term treatment with an agent is required and which would normally require frequent administration (e.g. at least daily injection).

It will be appreciated that the amount of an agent that is required is determined by biological activity and bioavailability which in turn depends on the mode of administration, the physicochemical properties of the agent employed and whether the agent is being used as a monotherapy or in a combined therapy. The frequency of administration will also be influenced by the above-mentioned factors and particularly the half-life of the agent within the subject being treated.

Optimal dosages to be administered may be determined by those skilled in the art, and will vary with the particular agent in use, the strength of the preparation, the mode of administration, and the advancement of the disease condition. Particularly, dosages may be determined with reference to the size of a tumour to be treated and/or with reference to the number of tumours to be treated. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of agents according to the invention and precise therapeutic regimes (such as daily doses of the compounds and the frequency of administration).

Generally, a daily dose of between 0.01 μg/kg of body weight and 1.0 g/kg of body weight of agents according to the invention may be used to prevent and/or treat cancer, depending upon which specific agent is used. More preferably, the daily dose is between 0.01 mg/kg of body weight and 100 mg/kg of body weight.

Daily doses may be given as a single administration (e.g. a single daily injection or oral administration). Alternatively, the agent used may require administration twice or more times during a day. As an example, agents according to the invention may be administered as two (or more depending upon the severity of the cancer to be treated) daily doses of between 10 μgs and 5000 mgs in injection form. A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

In accordance with the first, second or third aspects of the invention, a suitable amount of the agent of the invention capable of preventing and/or treating cancer may comprise from about 0.01 mg to about 800 mg. In another embodiment, the amount of the agent is an amount from about 0.01 mg to about 500 mg. In another embodiment, the amount of the agent is an amount from about 0.01 mg to about 250 mg. In another embodiment, the amount of the agent is an amount from about 0.1 mg to about 100 mg. In another embodiment, the amount of the agent is an amount from about 0.1 mg to about 20 mg.

Administration of 1-25 μg/ml of a class III SLRP such as opticin has been found to be particularly effective for prevention and/or treatment of cancer. Class III SLRPs such as opticin may be administered at a concentration of 1-25 μg/ml, and preferably at a concentration of 1-10 μg/ml, or even 1-5 μg/ml.

It will be appreciated that preferred doses of class III SLRPs other than opticin may be determined using similar methods to those employed in the Examples, but it is envisaged that administration of these compounds at a concentration of 1-25 μg/ml may achieve a therapeutic effect.

In accordance with the second aspect of the invention, the agent capable of promoting class III SLRP activity may be administered in the form of a pharmaceutical composition comprising a therapeutically effective amount of an agent according to the invention and a pharmaceutically acceptable vehicle. A “therapeutically effective amount” is any amount of an agent according to the invention which, when administered to a subject is able to prevent and/or treat cancer. A “subject” is a vertebrate, mammal, domestic animal or human being.

A “pharmaceutically acceptable vehicle” is referred to herein is any physiological vehicle known to those of ordinary skill in the art useful in formulating pharmaceutical compositions.

In a preferred embodiment, the pharmaceutical vehicle is a liquid and the pharmaceutical composition is in the form of a solution. In another embodiment, the pharmaceutically acceptable vehicle is a solid and the composition is in the form of a powder or tablet. In a further embodiment, the pharmaceutical vehicle is a gel and the composition is in the may be in the form of a cream or the like.

A solid vehicle can include one or more substances which may also act as flavoring agents, lubricants, solubilizers, suspending agents, fillers, glidants, compression aids, binders or tablet-disintegrating agents; it can also be an encapsulating material. In powders, the vehicle is a finely divided solid that is in admixture with the finely divided active agent. In tablets, the agent is mixed with a vehicle having the necessary compression properties in suitable proportions and compacted in the shape and size desired. The powders and tablets preferably contain up to 99% of the active agent. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles are used in preparing solutions, suspensions, emulsions, syrups, elixirs and pressurized compositions. The active agent can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal, subcutaneous and particularly intravenous injection. The compounds may be prepared as a sterile solid composition that may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

Agents according to the invention can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

Agents according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated by the skilled person that agents suitable for use according to the invention also include agents encoding class III SLRPs (as well as their derivatives). For example, possible agents include nucleic acid molecules encoding class III SLRPs. Such nucleic acids may preferably be administered in suitable vectors.

Accordingly it will be appreciated that the present invention provides the use of a vector encoding a molecule selected from the group comprising:

a) opticin; or

b) epiphycan; or

c) mimecan (also known as osteoglycin); or

d) a chimeric molecule comprising elements of any of a) to c);

e) a modified form of any of a) to d); or

f) a biologically active fragment or derivative of any of a) to e)

as an agent that promotes class III SLRP activity for use in the manufacture of a medicament for the prevention and/or treatment of cancer. It will further be appreciated that such vectors may provide suitable agents for use in gene therapy.

An example of a vector suitable for use in accordance with this embodiment comprises opticin cDNA inserted into a plasmid containing the E1/E3 deleted Ad5 genome (i.e. an adenovirus-derived vector). The inventors have found that this vector (termed “adeno-opticin”) is effective in the prevention or inhibition of tumour growth in vivo. Adeno-opticin may be provided at multiplicities of infection (MOI, an index of the ratio of infectious virus particles to cells) of at least 10, preferably at least 20, more preferably at least 50, even more preferably at least 100, and most preferably greater than 100.

The inventors have also found that intratumoural administration of adeno-opticin (for example by injection at a concentration of 10⁸ plaque forming units per 200 mm³ tumour volume) is able to prevent or inhibit tumour growth and/or progression. Although single intratumoural administrations of agents of the invention may be effective in the treatment of cancer it may be preferred that multiple administrations are effected. The number and duration of such administration may be determined by a responsible clinician, but may involve repeated administration at intervals of 2, 3, 4 or more days.

A convenient way in which cancer may be prevented and/or treated is to provide a therapeutically effective amount of such an agent in accordance with the invention at a site where such activity is required by means of gene therapy. In accordance with this embodiment of the invention the agent may preferably comprise a vector as described in the preceding paragraphs.

According to a fourth aspect of the present invention there is provided a delivery system for use in a gene therapy technique, said delivery system comprising a nucleic acid molecule encoding for an agent in accordance with the invention, said nucleic acid molecule being capable of bringing about expression of the chosen agent. Preferably the nucleic acid molecule is a DNA molecule capable of being transcribed to lead to the expression of the chosen agent.

According to a fifth aspect of the present invention there is provided the use of a delivery system as defined in the preceding paragraph for use in the manufacture of a medicament for use in the prevention and/or treatment of cancer.

According to a sixth aspect of the present invention there is provided a method of preventing and/or treating cancer, the method comprising administering to a patient in need of such prevention and/or treatment a therapeutically effective amount of a delivery system as defined for the fourth aspect of the invention.

Due to the degeneracy of the genetic code, it is clear that nucleic acid sequences encoding agents suitable for use in accordance with the invention may be varied or changed without substantially affecting the sequence of the product encoded thereby, to provide a functional variant thereof. An agent suitable for use in accordance with the invention must retain the ability to prevent and/or treat cancer, for example by inhibiting cancer cell proliferation and/or viability, and/or promoting cancer cell apoptosis.

Suitable nucleotides encoding agents in accordance with the invention (including class III SLRPs, fragments and derivatives thereof) include those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The delivery systems according to the invention are highly suitable for achieving sustained levels of an agent in accordance with the invention at a site where it is wished to prevent and/or treat cancer over a longer period of time than is possible for most conventional delivery systems. For example, agents in accordance with the invention suitable for the prevention and/or treatment of cancer may be continuously expressed (preferably at the site where at the site where cancer is to be treated) by cells that have been transformed with the nucleic acid molecule disclosed in respect of the fourth aspect of the invention. Therefore, even if the agent in accordance with the invention has a very short half-life in vivo, therapeutically effective amounts of the agent may be continuously expressed from the treated tissue.

Furthermore, the delivery system of the invention may be used to provide the nucleic acid molecule (and thereby the agent in accordance with the invention) without the need to use conventional pharmaceutical vehicles such as those required in ointments or creams that may otherwise be used in accordance with the invention.

The delivery system of the present invention is such that the nucleic acid molecule is capable of being expressed (when the delivery system is administered to a patient) to produce an agent in accordance with the invention which directly or indirectly has activity for the prevention and/or treatment of cancer. By “directly” we mean that the product of gene expression per se has the required activity. By “indirectly” we mean that the product of gene expression undergoes or mediates (e.g. as an enzyme) at least one further reaction to provide an active agent having the requisite activity.

The nucleic acid molecule may be contained within a suitable vector to form a recombinant vector. The vector may for example be a plasmid, cosmid or phage. Such recombinant vectors are highly useful in the delivery systems of the invention for transforming cells with the nucleic acid molecule.

Recombinant vectors may also include other functional elements. For instance, recombinant vectors may be designed such that the vector will autonomously replicate in the nucleus of the cell. In this case, elements that induce DNA replication may be required in the recombinant vector. Alternatively the recombinant vector may be designed such that the vector and recombinant DNA molecule integrates into the genome of a cell. In this case DNA sequences which favour targeted integration (e.g. by homologous recombination) are desirable. Recombinant vectors may also have DNA coding for genes that may be used as selectable markers in the cloning process.

The recombinant vector may also further comprise a promoter or regulator to control expression of the gene as required.

A DNA molecule suitable for use in accordance with the invention may (but not necessarily) be one that becomes incorporated in the DNA of cells of the subject being treated. Undifferentiated cells may be stably transformed leading to the production of genetically modified daughter cells. When this is the case, regulation of expression in the subject may be required e.g. with specific transcription factors, gene activators or more preferably with inducible promoters which transcribe the gene in response to a signal specifically found at a tumour or other site at which cancer to be prevented and/or treated are located. Alternatively, the delivery system may be designed to favour unstable or transient transformation of differentiated cells in the subject being treated. In this instance, regulation of expression may be less important because expression of the DNA molecule will stop when the transformed cells die or stop expressing the protein (ideally when the cancer has been successfully prevented and/or treated).

The delivery system may provide the nucleic acid molecule to a subject without it being incorporated in a vector. For instance, the nucleic acid molecule may be incorporated within a liposome or virus particle. Alternatively the “naked” nucleic acid molecule may be inserted into a subject's cells by a suitable means e.g. direct endocytotic uptake.

The nucleic acid molecule may be transferred to the cells of a subject to be treated by transfection, infection, microinjection, cell fusion, protoplast fusion or ballistic bombardment. For example, transfer may be by ballistic transfection with coated gold particles, liposomes containing the nucleic acid molecule, viral vectors (e.g. adenovirus as contemplated above) and means of providing direct nucleic acid uptake (e.g. endocytosis), for example by application of plasmid DNA directly (for instance either topically or by injection) to a site where cancer is to be prevented and/or treated.

The agent in accordance with the invention expressed from the DNA molecule may be a class III SLRP, or a biologically active fragment or derivative thereof.

Methods of the invention may be put into practice by inducing increased cellular expression of an agent in accordance with the invention, which may then bring about the required prevention and/or treatment of cancer. Such therapeutic expression of an agent in accordance with the invention may be achieved by increasing naturally occurring expression of the agent (for example the natural expression of a naturally occurring class III SLRP), or by inducing unnatural expression of the agent (e.g. induction of class III SLRP expression by cells that do not naturally express class III SLRPs) or by inducing over-expression of the agent. It will be appreciated that an increase in endogenous expression of class III SLRPs such as opticin may readily be achieved by the administration of an agent capable of increasing the transcription of the gene encoding opticin.

According to a seventh aspect of the invention there is provided a method of screening a test compound for its ability to prevent and/or treat cancer, the method comprising:

i) assessing the degree of binding between a class III SLRP and a substrate of the class III SLRP in the absence of the test compound,

ii) assessing the degree of binding between the class III SLRP and the substrate in the presence of the test compound,

iii) comparing the degree of binding occurring in step i) with the degree of binding occurring in step ii);

wherein a test compound is able prevent and/or treat cancer if the degree of binding occurring in step ii) is less than the degree of binding occurring in step i).

Compounds identified using the method of screening of the invention may be expected to inhibit cancer cell proliferation and/or viability, and/or to promote cancer cell apoptosis.

The substrate may be heparin or heparan sulphate, or a growth factor (particularly a growth factor such as EGF or FGF) or a growth factor receptor (particularly an EGF receptor or FGF receptor). Alternatively the substrate may comprise a cancer cell of a type associated with a cancer to be prevented and/or treated. The substrate may be a cellular receptor, and preferably a cellular receptor expressed by cancer cells, bound by class III SLRPs. Examples of preferred receptors include the cellular receptors for VEGF and FGF.

It will be appreciated that other agents having the same cellular receptor-binding profile as class III SLRPs, such as opticin, may be expected to have similar biological effects to those exhibited by class III SLRPs. Thus, in a eighth aspect of the present invention there is provided the use of an agent having substantially the same receptor-binding profile as a class III SLRP in the manufacture of a medicament for the prevention and/or treatment of cancer.

It will also be appreciated that the recognition of the importance of the receptor-binding profile of class III SLRPs in mediating the biological behaviour of these molecules provides a means by which agents able to mimic class III SLRP activity (for example by inhibiting cancer cell proliferation and/or viability, and/or promoting cancer cell apoptosis) may be identified.

Accordingly, the invention provides a method of screening a test compound for the ability to mimic class III SLRP activity, the method comprising comparing the receptor-binding profile of the test compound with the receptor-binding profile of a class III SLRP, wherein having substantially the same receptor-binding profile as a class III SLRP indicates that the class III SLRP is able to mimic class III SLRP activity.

The characteristic receptor-binding profiles of class III SLRPs may be readily determined using experimental methods and protocols well known to those skilled in the art.

The invention will be further described in the following Examples, with reference to the accompanying drawings in which:

FIG. 1 represents the full-length amino acid sequences of mature human and bovine opticin, human epiphycan and human mimecan, as well as illustrating amino acid alignment between the leucine-rich repeat regions of the human class III SLRPs;

FIG. 2 illustrates in Panel 2a the amino acid sequence of an NH terminal fragment of bovine opticin that is released on digestion with the metalloproteinases MMP2 or MMP9, in Panel 2b the amino acid sequence of preferred fragments of human and bovine opticin, and in Panel 2c amino acid alignment between the NH terminal opticin sequences from different species;

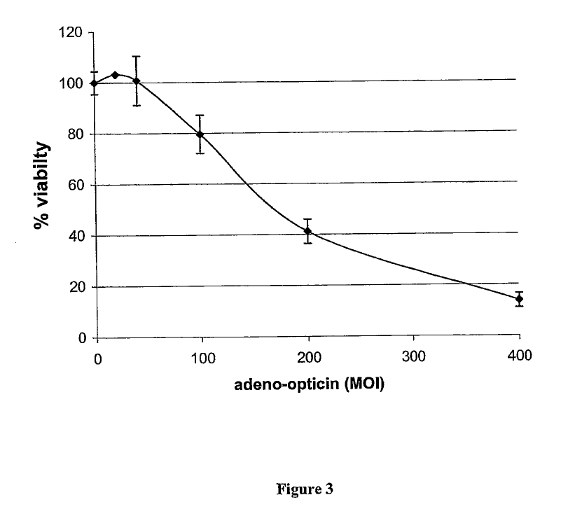

FIG. 3 illustrates the effect of infection (using increasing MOIs) of viral vectors encoding the class III SLRP opticin on tumour cell viability in HT1080 cells derived from fibrosarcoma;

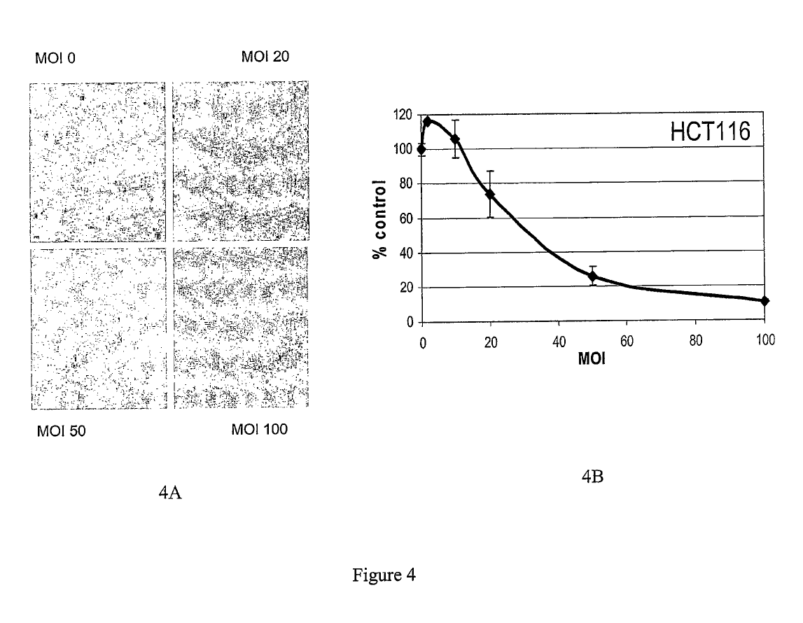

FIG. 4 illustrates the effect of infection (using increasing MOIs) of viral vectors encoding the class III SLRP opticin on tumour cell viability in HCT116 cells derived from colon cancer. Panel 4A shows micrographs of cultured HCT116 cells subject to infection with increasing MOI, whilst panel 4B depicts the data in the form of a graph charting decreased viability versus increased infection;

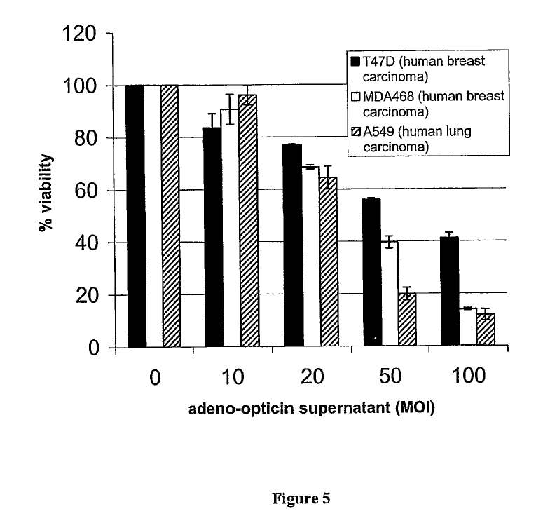

FIG. 5 is a bar chart illustrating the effect of treatment with the secreted class III SLRP opticin on viability of a number of diverse cancer cell lines;

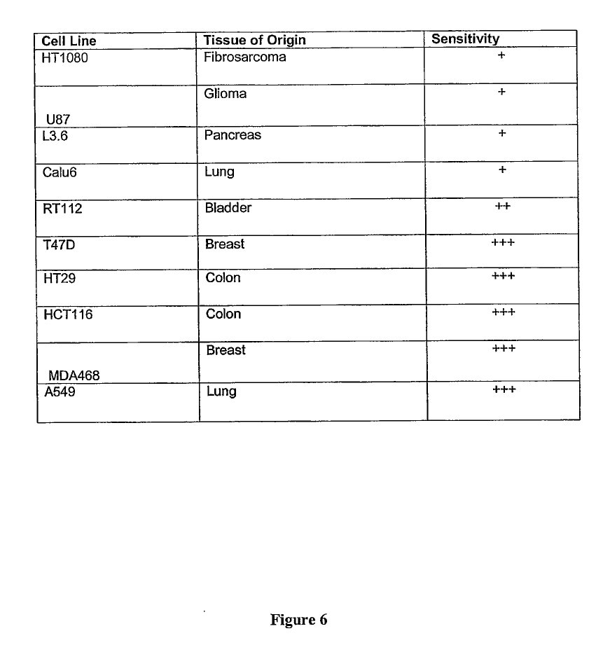

FIG. 6 is a Table illustrating the effect of treatment with the secreted recombinant class III SLRP opticin on viability of an increased number of cancer cell lines;

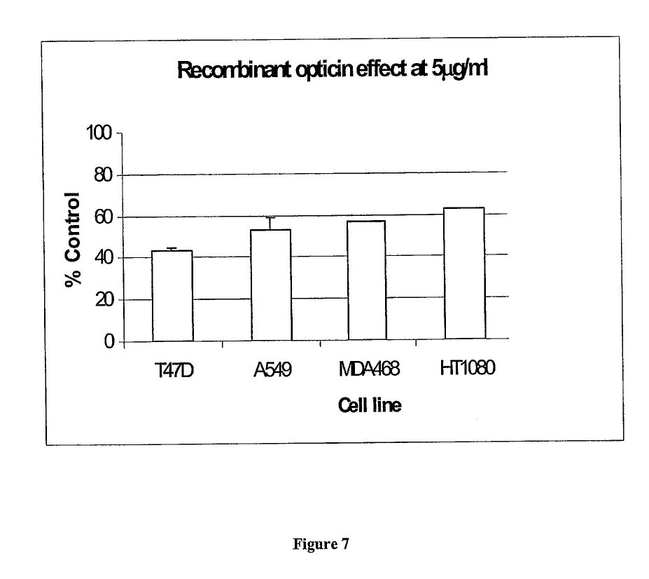

FIG. 7 is a bar chart illustrating the effect of treatment with the purified recombinant class III SLRP opticin (at a concentration of 5 μg/ml) on viability of a number of diverse cancer-derived cell lines; and

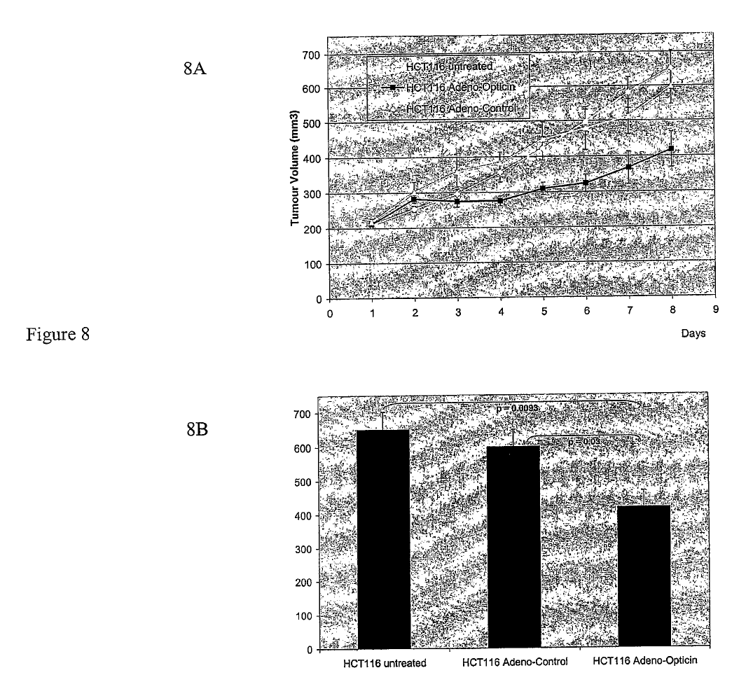

FIG. 8 illustrates the effect of intratumoural injection of a vector encoding the class III SLRP opticin on the volume of HCT116-based tumours in a xenograft model. Panel 7A shows the progression of the volume of treated, control and untreated tumours over time, whilst panel 7B compares the volume of treated, control and untreated tumours after eight days.

### EXAMPLES

The effects of class III SLRPs on cancer cells were investigated by the following experimental Examples. The experiments outlined below illustrate the effect on cancer cells of treatment with:
i) viral vectors encoding class III SLRPs;
ii) class III SLRPs expressed and secreted by cells infected with the viral vectors; and
iii) purified recombinant class III SLRPs.
   1. Effect of Infection with Viral Vector Encoding Class III SLRP—Part I.

The effects on cancer cells of infection with viral vectors encoding class III SLRPs were investigated using a first generation E1/E3 deleted adenovirus type 5 vector encoding for constitutive expression of the bovine class III SLRP opticin (a vector termed “adeno-opticin”). The corresponding “empty” vector was used as a control to establish that observed effects were attributable to the activity of the encoded class III SLRPs.

Cells of the human fibrosarcoma cell line HT1080 were infected with the adeno-opticin vector at a number of different multiplicities of infection (MOI, an index of the ratio of infectious virus particles to cells), and the effects of this infection on cell viability measured at three days post-infection. The results of this investigation are shown in FIG. 3.

FIG. 3 clearly illustrates that infection of HT1080 tumour cells with the adeno-opticin vector brought about a reduction in cell proliferation and an increase in cell death that lead to a significant decrease in cell viability. Increasing loss of viability was observed with increasing MOI, and an MOI of 400 (the highest dose tested) was able to bring about an 80% loss in tumour cell viability.

2. Effect of Infection with Viral Vector Encoding Class III SLRP—Part II.

The effects on cancer cells of infection with viral vectors encoding class III SLRPs were further investigated with reference to the colon cancer-derived cell line HCT116.

HCT116 cells were cultured and subject to infection with increasing MOIs of the adeno-opticin vector described above. The results of this study are shown in FIG. 4. Panel A of FIG. 4 shows micrographs of cultured HCT116 cells infected with adeno-opticin at MOI of 0, 20, 50 and 100. These micrographs illustrate that increasing the MOI of the adeno-opticin applied to the cells leads to increased “rounding” of the cells, which is in turn indicative of decreased cell viability. Indeed, in the micrograph showing HCT116 cells infected with adeno-opticin at an MOI of 100 very few viable cells are found to be present.

These results are also illustrated in the graph shown in Panel B of FIG. 4, which also shows that increasing infection of HCT116 cells leads to decreasing viability in the infected cells (as compared to viability in control cell populations).

3. Effect of Treatment with Class III SLRPs Expressed and Secreted by Cells Infected with Viral Vector—Part I.

Class III SLRPs such as opticin are secreted by cells in which they are expressed. The following study investigated the effects on cancer cells of opticin expressed by a population of cells other than the cancer cells to be treated.

The opticin-containing supernatant from the adeno-opticin infected HT1080 cells described above was subjected to Western blotting using the opticin-specific antibody OPT-A (generated by the inventors and described in “Le Goff et al. J. Biol. Chem. 2003: 278: 45280-45287”) to confirm the presence of opticin in the media.

Opticin-containing supernatant collected as set out above overlaid on three different tumour cell lines. The selected cell lines comprised a lung carcinoma cell line (A549) and two breast carcinoma cell lines (T47D and MDA468). The effects of opticin on the viability of these cancer cell lines are illustrated in FIG. 5.

FIG. 5 illustrates that tumour cell lines derived from diverse tissue backgrounds showed marked inhibition of growth in the presence of the class III SLRP opticin. These results indicate both that class III SLRPs are able to prevent and/or treat cancer derived from a number of tissues, and also that the viability of cancer cells may be inhibited by class III SLRPs expressed and secreted by cells other than the cells to be treated.

4. Effect of Treatment with Class III SLRPs Expressed and Secreted by Cells Infected with Viral Vector—Part II.

In an expansion of the study detailed above opticin-containing supernatants were overlaid on an increased number of cancer-derived cell lines. The cell lines used in this expanded study comprised cells derived from fibrosarcoma (HT1080), glioma (U87), pancreatic cancer (L3.6), lung cancer (Calu6 and A549), bladder cancer (RT112), breast cancer (T47D and MDA468) and colon cancer (HT29 and HCT116).

The cell lines listed above were incubated with the opticin-containing supernatants (produced by infection of HT1080 cells with adeno-opticin at an MOI of 100) for a period of 72 hours.

The results of this study are set out in the Table of FIG. 6, in which cell lines are arranged in order of their sensitivity to the class III SLRP opticin (as determined by MTT assay). In the Table “+++” denotes greater than 50% inhibition, “++” denotes greater than 25% inhibition, and “+” denotes 10-25% inhibition.

It will be noted that all cancer-derived cell lines tested were sensitive to the anti-proliferative effects of the class III SLRP opticin. Furthermore, a subset of cell lines tested exhibited particularly marked sensitivity to the anti-proliferative effects of the class III SLRP opticin. These include cells derived from cancers of the colon (HCT116, HT29), breast (MDA468, T47D) and lung (A549), and accordingly these cancers represent particularly preferred cancers for treatment in accordance with the present invention.

5. Effect of Treatment with Purified Recombinant Class III SLRPs.

The effect of treatment of cancer cells with purified recombinant class III SLRPs was investigated as follows.

Four cancer cell lines (T47D, A549, MDA468 and HT1080 as described above) were incubated with the purified recombinant class III SLRP opticin. The effect of opticin on the proliferation and viability of these cells is illustrated in FIG. 7.

FIG. 7 illustrates that treatment with the recombinant class III SLRP opticin was able to inhibit the proliferation and viability of four cell lines derived from diverse tumour types. Calculation based on these data indicated that the IC50 was approximately 5 μg/ml of opticin. The results illustrate that recombinant purified class III SLRPs are able to effectively prevent and/or treat cancers in a number of tissue types.

6. Effect of Class III SLRPs on Tumour Growth In Vivo.

The ability of class III SLRPs to directly inhibit tumour growth and proliferation in vivo was investigated using a xenograft model.

HCT116 cells were implanted intra-dermally into nude mice and tumours allowed to grow to a size of ˜200 mm³. This size is known to constitute a palpable tumour mass and it can be predicted that if left untreated the tumour formed will double in volume (termed relative tumour volume RTV2) by day 4-5 and reach RTV3 by day 8-9.

Adeno-opticin was administered to experimental HCT116 tumours (n=6; treatment size range 192-225 mm³) as a single intratumoural injection of 10⁸ plaque forming units. Since the adeno-opticin vector is non-integrative it can be predicted that a peak of opticin expression by infected cells will occur 24-36 hours after injection, followed by a decline in expression thereafter.

The results of this study are shown in FIG. 8, and illustrate that treatment of in vivo tumours with even a relatively modest dose of the viral vector adeno-opticin is sufficient to significantly inhibit tumour progression.

Panel A of FIG. 8 illustrates the volumes (in mm³) of HCT116 tumours treated with class III SLRP opticin via the viral vector adeno-opticin (black squares), the “empty” viral vector (“adeno-control” shown with white triangles) and untreated tumours (white diamonds). It can be seen that a single treatment with opticin initially halts tumour growth (over days 2 to 4), and then significantly inhibits tumour growth over days 5 to 8.

This result is further shown in Panel B of FIG. 8 which illustrates that at day 8 (by which control tumours had undergone a three-fold expansion “RTV3”) opticin treated tumours were 52% smaller than controls. This reduction in HCT116 tumour growth caused by opticin treatment results in a significant 5.8 day growth delay compared to untreated controls (p=0.005 as determined by non-parametric Mann-Whitney U Test).

Summary.

The experimental results provided above illustrate that class III SLRPs such as opticin may prevent and or treat cancers derived from a number of different tissues by directly inhibiting proliferation and/or viability of the cancer cells.

The data indicate that effective prevention and/or treatment of cancer using class III SLRPs may be brought about through at least four mechanisms as follows.

i) Cancer may be prevented and/or treated by expression of class III SLRPs such as opticin by the cancer cells to be treated. Such expression may suitably be induced by the use of vectors encoding the chosen class III SLRP.

ii) Cancer may be prevented and/or treated by class III SLRPs expressed and secreted by cells other than the cancer cells to be treated. It will be appreciated that the expression of the class III SLRP may be induced in cells located in the area surrounding the cancer to be prevented and/or treated, or that the expression may be induced at a site distant to the cancer provided that the expressed and secreted class III SLRP is subsequently able to influence the activity of the cancer cells (e.g. by inhibiting proliferation and/or viability of the cancer cells, and/or stimulating their apoptosis).

iii) Cancer may be prevented and/or treated by exposure of cancer cells to purified recombinant class III SLRPs. Such exposure may be brought about by injection or other introduction of the purified recombinant class III SLRP into a tumour to be treated.

iv) Cancer may be prevented and/or treated by intratumoural administration of class III SLRPs or agents capable of inducing the expression of class III SLRPs. The results illustrate that this is particularly effective in the inhibition or prevention of tumour growth and/or progression.

The skilled person will readily appreciate that as an alternative to the introduction of or expression of class III SLRPs themselves, small molecules based on class III SLRPs (such as peptide or other pharmacologic derivatives) could be introduced or expressed in order to produce the same effects. The market for anti-cancer agents is very large and there is much interest in using naturally occurring molecules in cancer treatment because of the low risks of toxicity.

There is also considerable interest in the use of virally-delivered agents having anti-cancer activity in the treatment of tumours, and the results presented above clearly illustrate that class III SLRPs represent suitable examples of such agents capable of use in the prevention and/or treatment of cancer.

1-23. (canceled)

24. A method of inhibiting proliferation or viability of cancer cells comprising contacting the cells with an amount of an agent that promotes class III SLRP activity, the amount effective to inhibit proliferation or reduce viability of the cells.

25. The method according to claim 24, wherein the cancer cells are a tumor cells.

26. The method according to claim 25, wherein the tumor cells are an avascular tumor cells.

27. The method according to claim 24, wherein the cancer cells are a fibrosarcoma cells.

28. The method according to claim 25, wherein the cancer cells are a fibrosarcoma cells.

29. The method according to claim 26, wherein the cancer cells are a fibrosarcoma cells.

30. The method according to claim 24, wherein the cancer cells are breast cancer cells.

31. The method according to claim 25, wherein the cancer cells are breast cancer cells.

32. The method according to claim 26, wherein the cancer cells are breast cancer cells.

33. The method according to claim 24, wherein the cancer cells are lung cancer cells.

34. The method according to claim 25, wherein the cancer cells are lung cancer cells.

35. The method according to claim 26, wherein the cancer cells are lung cancer cells.

36. The method according to claim 24, wherein the cancer cells are glioma cells.

37. The method according to claim 25, wherein the cancer cells are glioma cells.

38. The method according to claim 26, wherein the cancer cells are glioma cells.

39. The method according to claim 24, wherein the cancer cells are pancreatic cancer cells.

40. The method according to claim 25, wherein the cancer cells are pancreatic cancer cells.

41. The method according to claim 26, wherein the cancer cells are pancreatic cancer cells.

42. The method according to claim 24, wherein the cancer cells are bladder cancer cells.

43. The method according to claim 25, wherein the cancer cells are bladder cancer cells.

44. The method according to claim 26, wherein the cancer cells are bladder cancer cells.

45. The method according to claim 24, wherein the cancer cells are colon cancer cells.

46. The method according to claim 25, wherein the cancer cells are colon cancer cells.

47. The method according to claim 26, wherein the cancer cells are colon cancer cells.

48. The method according to claim 24, wherein the agent is a class III SLRP agent.

49. The method according to claim 24, wherein the agent is selected from the group consisting of:
a) opticin;
b) epiphycan;
c) mimecan;
d) a chimeric molecule comprising elements of a), b) or c);
e) a modified form of any of a), b), c), or d); and
f) a biologically active fragment or derivative of a), b), c), d) or e).

50. The method according to claim 49, wherein the biologically active fragment is an N-terminal fragment.

51. The method according to claim 49, wherein the biologically active fragment is a leucine-rich repeat fragment.

52. The method according to claim 49, wherein the derivative has a half-life in vivo greater than the half-life in vivo of the agent from which it is derived.

53. The method according to claim 52, wherein the derivative is a peptoid derivative.

54. The method according to claim 52, wherein the derivative is a D-amino acid derivative.

55. The method according to claim 49, wherein the derivative is a peptide-peptoid hybrid derivative.

56. The method according to claim 24, wherein the agent is a vector encoding a molecule selected from the group consisting of:
a) opticin;
b) epiphycan;
c) mimecan;
d) a chimeric molecule comprising elements of any of a), b) or c);
e) a modified form of any of a), b), c) or d); and f) a biologically active fragment or derivative of any of a), b), c), d) or e).

57. The method according to claim 56, wherein the biologically active fragment or derivative is selected from the group consisting of an N-terminal fragment; a leucine-rich repeat fragment; a derivative that has a half-life in vivo greater than the half-life in vivo of the agent from which it is derived; a peptoid derivative; a D-amino acid derivative; a peptide-peptoid hybrid derivative and combinations thereof.

58. The method according to claim 56, wherein the vector is an adenoviral vector encoding opticin.

59. The method according to claim 24 wherein the agent promotes or increases cancer cell apoptosis.

60. The method according to claim 24, wherein an amount of the agent is administered to a subject having cancer, which amount is effective to inhibit proliferation or reduce viability of the cells.

61. The method according to claim 60, wherein the amount is a daily dose of between 0.01 μg/kg and 1 g/kg.

62. A method of inhibiting proliferation or viability of cancer cells, comprising contacting the cells with an agent that promotes class III SLRP activity and an anti-cancer agent.


SEQUENCE LISTING (14 sequences)

SEQ ID NO: 1 - protein, 313 aa, Homo sapiens
ASLPRKERKRREEQMPREGDSFEVLPLRNDVLNPDNYGEVIDLSNYEELTDYGDQLPEVKVTSLAPATSISPAKSTTAPGTPSSNPTMTRPTTAGLLLSSQPNHGLPTCLVCVCLGSSVYCDDIDLEDIPPLPRRTAYLYARFNRISRIRAEDFKGLTKLKRIDLSNNLISSIDNDAFRLLHALQDLILPENQLEALPVLPSGIEFLDVRLNRLQSSGIQPAAFRAMEKLQFLYLSDNLLDSIPGPLPLSLRSVHLQNNLIETMQRDVFCDPEEHKHTRRQLEDIRLDGNPINLSLFPSAYFCLPRLPIGRFT

SEQ ID NO: 2 - protein, 302 aa, Bos sp.
ASLSEEREGDPYAILHLGDYVLSLDNYDEVIDPSNYDELIDYGDQLPQVKGTSLASLTRTRFTQSTEAARTLPSNPTTARPPTLGLLAAPANHGLPTCLICVCLGSSVYCDDADLENIPPLPQTTAYLYARFNRISHIRAGDFKGLTKLKRIDLSGNSISSIDDKALRLLPALRDLILPENKLVALPTLPTSIEVLDVRMNRLQSSGIQPEAFRALEKLQFLYLADNLLDAIPPSLPLSLRSLHLQNNMIETMQRDAFCDAEEHRHTRRPLEDIRLDGNPINLSLFPSAYFCLPRLPTGRFV

SEQ ID NO: 3 - protein, 300 aa, Homo sapiens
APTLESINYDSETYDATLEDLDNLYNYENIPVGKVEIEIATVMPSGNRELLTPPPQPEKAQEEEEEEESTPRLIDGSSPQEPEFTGVLGPHTNEDFPTCLLCTCISTTVYCDDHELAIPPLPKNTAYFYSRFNRIKKINKNDFASLSDLKRIDLTSNLISEIDEDAFRKLPQLRELVLRDNKIRQELPTTLTFIDISNNRLGRKGIKQEAFKDMYDLHHLYLTDNNLDHIPLPLPENLRALHLQNNNILEMHEDTFCNVKNLTYIRKALEDIRLDGNPINLSKTPQAYMCLPRLPVGSLV

SEQ ID NO: 4 - protein, 281 aa, Homo sapiens
KPAPPTQQDSRIIYDYGTDNFEESIFSQDYEDKYLDGKNIKEKETVIIPNEKSLQLQKDEAITPLPPKKENDEMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 5 - protein, 209 aa, Homo sapiens
GLPTCLVCVCLGSSVYCDDIDLEDIPPLPRRTAYLYARFNRISRIRAEDFKGLTKLKRIDLSNNLISSIDNDAFRLLHALQDLILPENQLEALPVLPSGIEFLDVRLNRLQSSGIQPAAFRAMEKLQFLYLSDNLLDSIPGPLPLSLRSVHLQNNLIETMQRDVFCDPEEHKHTRRQLEDIRLDGNPINLSLFPSAYFCLPRLPIGRFT

SEQ ID NO: 6 - protein, 209 aa, Homo sapiens
DFPTCLWCTCISTTVYCDDHELDAIPPLPKNTAYFYSRFNRIKKINKNDFASLSDLKRIDLTSNLISEIDEDAFRKLPQLRELVLRDNKIRQLPELPTTSTFIDISNNRLGRKGIKQEAFKDMYDLHHLYLTDNNLDHIPLPLPENLRALHLQNNNILEMHEDTFCNGKNLTYIRKALEDIRLDGNPINLSKTPQAYMCLPRLPVGSLV

SEQ ID NO: 7 - protein, 209 aa, Homo sapiens
EMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 8 - protein, 87 aa, Bos sp.
ASLSEEREGDPYAILHLGDYVLSLDNYDEVIDPSNYDELIDYGDQLPQVKGTSLASLTRTRFTQSTEAARTLPSNPTTARPPTLGLL

SEQ ID NO: 9 - protein, 24 aa, Homo sapiens
DNYGEVIDLSNYEELTDYGDQLPE

SEQ ID NO: 10 - protein, 24 aa, Bos sp.
DNYDEVIDPSNYDELIDYGDQLPQ

SEQ ID NO: 11 - protein, 90 aa, Bos sp.
ASLSEEREGDPYAILHLGDYVLSLDNYDEVIDPSNYDELIDYGDQLPQVKGTSLASLTRTRFTQSTEAARTLPSNPTTARPPTLGLLAAP

SEQ ID NO: 12 - protein, 96 aa, Canis sp.
ASLPKERKRRDEMHGEGDSYVVLGNYVLGPDNYDEVIDLSDYEGLMDYGDQLPEAKVTNLAPPTGISSAQSTMTPRTLSLKPTMIRPTELGVLGSP

SEQ ID NO: 13 - protein, 100 aa, Homo sapiens
ASLPRKERKRREEQMPREGDSFEVLPLRNDVLNPDNYGEVIDLSNYEELTDYGDQLPEVKVTSLAPATSISPAKSTTAPGTTSSNPTMTRPTTAGLLLSS

SEQ ID NO: 14 - protein, 97 aa, Mus sp.
ASLLGEREREEQSPEEGDTYASLYVGNHTLSIEDYNEVIDLSNYEELADYGDQIPEAKISNLTLPTRTSPTSTVAQKTLSPNLTMAVPTTTGLLNSQ